(19) United States
(12) Patent Application Publication
(10) Pub. No.: US 2004/0198840 A1
(43) Pub. Date: Oct. 7, 2004

(54) Hydrazide substrate safely shuts down disease activated protease to halt viral replication, cancerous cell division, and toxic protein generation

(76) Inventor: Reuben Edwin Deloach, Fort Worth, TX (US)

Correspondence Address: REUBEN E. DELOACH, 3916 LINKMEADOW DR., FORT WORTH, TX, 76008, US

(21) Appl. No.: 10/813,384

(22) Filed: Mar. 30, 2004

Related U.S. Application Data
(60) Provisional application No. 60/459,694, filed on Apr. 2, 2003.

(51) Int. Cl. A61K 31/15

(52) U.S. Cl. 514/664

(57) ABSTRACT

A hydrazide substrate is targeted by disease activated protease to shutdown protein products required by viral infections, cancer, and other diseases. Protease cleavage innately targets peptide bonds that are simulated in the hydrazide substrate molecule. Cleavage action releases the reactive hydrazine moiety that bonds to the protease enzyme structure causing its dysfunctional shutdown. That shutdown stops incessant disease activity that holds cell maintenance systems at bay, and also halts the production of proteins and peptides necessary for disease activity and proliferation as viral coat proteins and metastatic cancer proteins exemplify. Other uses for the hydrazide substrates mechanism also exists to shutdown protease systems innate to microorganisms as a way to halt production of peptide signals that induced cell division, growth, or reproduction of infectious organisms.

### CROSS REFERENCE TO RELATED APPLICATIONS

This application claims benefits of Provisional Application 60/459,694, filed Apr. 2, 2003.

### STATEMENT REGARDING FEDERALLY SPONSORED RESEARCH

(Not Applicable)

### BACKGROUND OF THE INVENTION

1. Statement of the Field of Endeavor

The field of endeavor to which this invention pertains is hydrazide substrate drugs as used in prior art applications to shutdown oxidase enzymes as an antidepressant method, and herewith such drugs are use to shutdown protease enzymes to halt protein production required by viral diseases and cancer.

2. Information Related to this Invention

This invention provides a new use for existing drugs of the mono amine oxidase enzyme inhibitor (MAOI) class that utilizes an irreversible substrate mechanism provided by a hydrazide substrate drug. Such drugs were discovered during the 1940's and 50's, and it appears there has been no increase of information or research interest in this field since that time. Information was not found by the applicant that provides a detailed mechanism of action, or hypothesis that could be used to explain the hydrazide substrate mechanism, and no effort appears to have been made in drug research activities to glean any undiscovered medical benefits for hydrazide substrate use since that time. As such this applicant provided the effort needed to discover the mechanism of action provided by the hydrazide substrate which is presented here to explain the mechanism of action utilized by prior art.

The mechanism of action provided by hydrazide drugs is based on hydrazide as an enzyme substrate material. The substrate is represented by an alkyl hydrazide molecule, or similar hydrazide molecule with a substituent attached to the hydrazine moiety. Such a molecule cannot react with organic substituents, but it does react to protease enzyme cleavage that targets such hydrazide substrate molecules. This is because such hydrazide substrate contains an amide type bond represented in the substrate molecule as a hydrazine to acid linkage (R′NHNHCOR″) that simulates the amide bond (R′NHCOR″) of a peptide chain. Peptide fragments are normally processed by protease as a resource of substrate material used in peptide assembly. However following protease cleavage of a single hydrazide molecule the enzyme is rendered dysfunctional because such substrate comprises a reactive hydrazine moiety which reacts to cleavage action by immediately attaching to the enzyme which obstructs further enzyme activity. Such cleavage action essentially transfers the hydrazine molecular bond from that of the substrate to that of the enzyme molecule causing the dysfunctional shutdown of the protease system and essentially rendering the cell inactive.

The hydrazide substrate shuts down active protease, and in particular disease activated protease to halt all disease related protein products. In the mammalian cell such static state induces cell maintenance action believed to survey cell genetic components by scanning the nucleotides to locate, repair, or replace dysfunctional or disease altered components, else apoptotic termination of the cell is initiated. Understanding this hydrazide mechanism and its ability to shutdown protease, and hence halt all protein produced by such cell engenders many novel uses as provided in the appropriate section of this disclosure. Such novel uses provided by hydrazide shutdown of protease is illustrated by the following facts:

[a] Without a protease system operational to supply viral coats protein for a viral infected cell, viral replication is completely halted;

[b] without the protease system operational to produce peptide signals that induce microorganism cell division, such organisms cannot divide, reproduce, or proliferate;

[c] without the protease system operational to produce protein and peptide signals that induce cancerous cell division, cancer cell division and metastasis is blocked;

[d] without the protease system operational to produce toxic and damaging proteins, and proteins necessary for metastasis, such diseases like myeloma and Alzheimer's disease are shutdown unable to proliferate or produce toxic protein that causes systemic damages;

[e] and the dysfunctional shutdown of incessant disease activated protease that holds cell maintenance systems at bay, also induces condition for cell maintenance action that is believed to either restore a healthy cell condition or to terminate the diseased cell completely.

Prior art provides examples of discoveries based on such facts as described above. For example Schoene and Hoffman in 1949, utilized maleic hydrazide to supply a hydrazide substrate to stop peptides that induced cell division and growth of plants; Fox in 1952, used isoniazid to supply a hydrazide substrate to stop peptides that induced cell division and reproduction of the tuberculosis organism; Zeller in 1952, used iproniazid to supply a hydrazide substrate to cause the dysfunctional shutdown of the oxidase enzyme system providing MAOI antidepressant effects; and Thompson in 1953, used semicarbazone derivatives to supply a hydrazide substrate to shutdown the protease system that supplied viral coat proteins to stop smallpox and polio virus action. These discoveries are further explained in the following:

Prior Art of Hydrazides to Stop Cell Division and Growth:

Science 109, 588 (1949), unveils Schoene and Hoffman's work related to a plant growth inhibitor as provided by a maleic hydrazide spray that is used to stop “suckering”, or new plant growth in tobacco farming, and in recent years such mechanism has been used to prevent biological changes as budding, ripening, and to retard spoilage of farm produce. Although no explanation for a mechanism of action has been found by this applicant listed in the literature, it is easy to understand based on the hydrazide substrate hypothesis presented here that maleic hydrazide provides a hydrazide substrate having a peptide like bond construction that will be targeted by plant cell protease enzyme cleavage causing the dysfunctional shutdown of the protease system. That shutdown will halt production of all peptides used to transmit biological changes which includes cell division, budding, ripening, and such like. Maleic hydrazide is a double acid hydrazide which is an inert, unreactive hydrazide molecule until the molecule is cleaved by enzyme action. When cleavage occurs the reactive hydrazine moiety is released which immediately bonds to the protease enzyme to halt any protein or peptide product that might initiate a biological change altering the status quo of the cell. As such the plant cell lives on without suffering any effects except it cannot undergo biological change. Such hydrazides have been in use for about 50 years and such use shows a safe history of use on grocery produce and tobacco plants.

Prior Art of Hydrazides to Stop Tuberculosis Infections:

Fox synthesized isoniazid in 1952 for use as a tuberculostatic agent, provided by the condensation of isonicotinic acid and hydrazine. In effect Fox discovered that this chemical functioned as a tuberculostatic agent that exceeded all other substances screened. Isoniazid was not of an inert hydrazide design like the embodiment of the present invention but had a predominating hydrazine characteristics that caused neurotoxic effects and metabolic disorders. This negative effect was because the exposed hydrazine radical is very reactive and could easily combine with numerous functional groups found throughout the body. It combines most easily with ester groups, ketones, and amides and as such is transformed in vivo to body. It combines most easily with ester groups, ketones, and amides and as such is transformed in vivo to various chemical compounds. However an antibacterial effect that would be lethal to tuberculosis organism was sought by Fox and to some extent that effect was provided by the toxic effect which was also injurious to the microorganism caused by the exposed hydrazine terminal.

The isoniazid molecule design was noted by Fox as being inactivated by systemic acetylation that also provided for the drug's rapid excretion found in about half the patients treated with isoniazid. This was because the reactive hydrazine terminal was exposed without an alkyl or other substituent that would have blocked its conjugation with esters and other body constituents such that the hydrazine toxic effect predominated, and as such it posed a high risk of autoimmune disease. The applicant believes this disadvantage was because the hydrazine terminal having no substituent attached could combine easily at the amide bond found in human protein, especially collagenous tissue, which could provoke an antigenic type response against such connective tissue which would develop into autoimmune disease. The need for a viable tuberculostatic agent apparently outweighed the risks and side effects provided by hydrazine and as such isoniazid was adopted for tuberculosis use which remains to be the principal tuberculostatic drug of all time.

Fox later tried the nontoxic alkylated form of isoniazid that negated the untoward toxic effects. That product was iproniazid, but iproniazid could not terminate the tuberculosis organism thereby motivating Fox to abandon iproniazid from consideration as a tuberculostatic agent. The current invention however shows that an inert hydrazide substrate drugs as iproniazid represents is effective to prevents the toxic effects caused by the exposed hydrazine terminal, and it is also effective at a small fraction of the dose required by isoniazid to prevent cell division and proliferation of any microorganisms. However iproniazid has no mechanism that can terminate or kill existing infectious organisms, or hasten their natural demise due to attrition. As such Fox was correct to assume that iproniazid was not a viable tuberculostatic agent.

Prior Art of Hydrazides as Oxidase Enzyme Inhibitor:

In 1952, Zeller began researching iproniazid's psychotropic effects following Fox's discovery that it raised the mood of persons given iproniazid during his testing on tuberculosis patients. As such Zeller's research indicated that iproniazid was an oxidase inhibitor, or more precisely was a mono amine oxidase inhibitor (MAOI) which explained the antidepressive effect reported. The following therapeutic success of iproniazid as an antidepressant motivated the development of a succession of oxidase enzyme inhibitors, or MAOI drugs. The MAOI mechanism was described as an irreversible substrate drug that shuts down the oxidase enzyme The oxidase enzyme normally provides for the destruction of biogenic amines, or neural stimulants, as norepinephrine, noradrenaline, and serotonin exemplify.

The regulation of biogenic amine stimulant levels are critical as excess accumulation in the CNS or brain tissue induces problematic behavior, and too little of such biogenic amines results in mental depression. As such an optimal level is provided by biological regulation schemes that activates oxidase enzymes as needed to destroy biogenic amines until the optimum level is reached. As such the oxidase enzyme system changes the stimulant into a non stimulant metabolite by oxidation. The applicant has shown that the mechanism of action is a two step process whereby the oxidase enzyme introduces oxygen to the alpha-carbon of the stimulant molecule to form an amide function. The applicant suggests that such transformation of an amine to an amide is sufficient to negate the neural stimulant effects of the biogenic amine. However a second step linked to the destruction of stimulants can be explained as the deamination of the metabolite.

Deamination requires targeted cleavage of the amide bond formed by oxidation necessary to remove the amino group. Because the hydrazide substrate provides a similar bond that is also target for such cleavage action it provides the irreversible substrate that halts the oxidase-demination processes. This can be explained as hydrazide substrate cleavage releases the hydrazine moiety of the substrate such that hydrazine attaches to the enzyme structure causing its dysfunctional shutdown. As a result oxidase enzyme activity and linked cell operations are halted which induces cell maintenance systems to activate which either repairs the damage caused by the hydrazine attachment to the enzyme, or either the cell is terminated or replaced. The benefit derived by such process is that the oxidase system is decommissioned for a space of time by the hydrazide substrate shutdown allowing biogenic amines to accumulate to a higher level before the oxidase system shutdown process is repeated. Such period without oxidase system destruction of biogenic amines allow such stimulants to increase in levels which provides the antidepressive effects characteristic of MAOI hydrazide substrate drugs.

Because the MAOI mechanism is an oxidase system shutdown is based on hydrazide substrate methods as provided by protease system shutdown based on hydrazide substrate method, it is noteworthy to recognize that a 50 year history of such hydrazide substrate drug use has existed without any untoward effects evolving from such long term use. Alkyl hydrazide drugs as prior art MAOI oxidase inhibitors, and as this invention provides to shutdown enzymes, is chemically inert and unable to react with body constituents, and only becomes active as a consequence of enzyme cleavage necessary to release the reactive hydrazine molecule that shuts down the enzyme. The process is essentially benign and without untoward side effects if MAOI drug precautions are observed as is necessary when using alkyl-hydrazide substrate drugs for any purpose.

Prior Art of Hydrazides to Stop Smallpox, Polio, and Other Viruses:

In about 1953, Thompson unknowing utilized the hydrazide mechanism as a smallpox antiviral agent which evolved as a product named marboran. He discovered antiviral action using isatin thiosemicarbazone molecules. Thompson showed that in large dose amounts such isatin thiosemicarbazones resisted vaccinia viral infections in mice but failed to recognize or attribute such effect to the hydrazide component that existed within the thiosemicarbazone molecule. Variations of the isatin molecule appeared to discriminate between different viruses. Methisazone, a derivative comprising an N-methyl derivative of the isatin moiety was reported to provide resistance to vaccinia and smallpox infections; a dimethyl derivative was active against mouse pox in the same limited fashion; and the dibutyl derivative appeared to have greater affinity for the poliovirus. The literature proposes that the drugs produced a defect in protein incorporation by interfering with attachment of mRNA to the ribosome. Based on an understanding of the hydrazine mechanism it is clear that the isatin thiosemicarbazone molecules provided a hydrazide substrate which was a target for protease action that shut down the production of all proteins needed for the viral replication process. As such necessary peptides and viral coat protein products were halted preventing viral replication.

The additional molecular structure that Thompson believed necessary to target different viruses was a mistake, as the hydrazide molecule is targeted by disease activated protease that exists in the infected cell. The least obstructive molecule design would make the most efficient target. As such any variance in efficacy was a reduction of efficacy and not an increase of efficacy. Such loss of efficacy was because of a loss of targeting provided by molecule size or shapes that can hide the amide like bond of hydrazide that is being targeted by protease action. As such Thompson failed to discover the hydrazide mechanism responsible for the antiviral effect that was optimum with a small hydrazide molecule, and could not be made more efficient by molecular additions, but was in fact made less efficient by such obstructions Had Thompson used a simple alkyl-hydrazide structure that would not have obstructed the innate targeting action of the viral activated protease he would have discovered an effective antiviral agent effective at 1/20th of the level required by the thiosemicarbazone product. As such Thompson's thiosemicarbazone antiviral agents were not of sufficient efficacy to satisfy the medical needs of his time and the marboran product faded from use.

Prior Art of Protease Inhibitors for HIV/AIDS Use:

Since the advent of HIV and the ensuing HIV/AIDS epidemic in the 1990's, a search for drugs that can control or limit replication of the HIV virus has been pursued vigorously. Any treatment scheme is complicated by fact that HIV has a particular affinity for the human T-4 lymphocyte cell surface which disrupts immune system functions. However all viruses have a common dependency on the protease system of infected cells that require production of protein viral coats and peptide components used to control and regulate biological processes in the infected cell. As such the dependency by HIV on the protease enzyme for viral related proteins necessary for HIV replication provides a vulnerable target which can be obstructed by protease inhibitor drugs or shutdown completely by hydrazide substrate drugs as the invention provides. A variety of such prior art drugs now exist that target specific receptors sites as would best serve to obstruct peptide assembly required by viral infections, or that at least would restrict overall protein production efficiency. Even at best such approach to prevent viral replication is inefficient as it serves to inhibit protein in a partial and temporary fashion, and the receptor site design method limits such application to a specific virus type and use. It does not use a method that can terminate protease action or shutdown the disease process as needed to eradicate the disease. The collateral damages affecting healthy cells is high which also contributes many untoward side effects and metabolic problems which is characteristic of such method.

Prior Art of Protease Inhibitor for Multiple Myeloma Use:

Protease inhibitor drugs are not only being used to treat HIV infections but are finding use in a number of cancer related applications. Since the existence and metastasis of myeloma is also dependent on protein or peptide products, any means that can intervene to limit or stop the protein products required by the disease provides a means to limit disease activity and proliferation. It is because protease inhibitors are able to slow the production of such peptide products and even reduce the amount of toxic proteins produced that such methodology is now being pursued to treat multiple myeloma. In 2002, Dr. K. Anderson of the Dana-Farber Cancer Institute published information in the Multiple Myeloma Foundation news letter that evidenced the successful results of such process based on restricting protease system activity using such technique. One such new proteasome inhibitor drug is Velcade (bortezomib).

### BRIEF SUMMARY OF THE INVENTION

General Idea of the Invention:

This general idea of this invention began with a curiosity raised by an entry in the Merck Index pertaining to maleic hydrazide as a plant growth inhibitor. If maleic hydrazide could stop cell division and growth of tobacco plants without causing harm to the plant as was stated, then why couldn't such biological mechanism be adapted to stop cancer cell division and growth for human use? Such biological changes as cell division are commonly induced by bioactive peptide signals, and surely the maleic hydrazide was inhibiting such peptides responsible for cell division. With some research and experimentation the hypothesis was proven correct and the general idea behind this invention was born.

In essence the idea was based on the fact that cancer and viral infections are dependent on protein and peptide products necessary for disease action and metastasis. Wy not use a MAOI hydrazide substrate drug like that used to shutdown oxidase enzyme systems, to shutdown protease enzyme systems? Such complete shutdown of protease by hydrazide would be far superior to the present method provided by protease inhibitor drugs which can only temporally block receptors to slowdown, or provide interference to the peptide assembly process.

### SUMMARY

Hydrazide substrate drugs have been used for more than 50 years to shutdown oxidase enzymes as MAOI use illustrates. It's unrecognized potential to shutdown protease enzymes as used by disease has apparently gone unrecognized. Its efficacy to be targeted by disease activated protease and to stop disease related protein is absolute, or 100%, whereas current protease inhibitor drugs can only inhibit a part of such protein being produced. The scope of diseases treatable by the hydrazide method is not limited to a single disease as HIV or myeloma, but includes all viruses, all cancers, and all other diseases that infect the cell or its DNA. Its action is not plagued with side effects and metabolic problems, but its action is rapid and without untoward side effects as the MAOI drug history of hydrazide substrate use shows.

The mechanism used to accomplish a shutdown of disease infected cells is derived from the inert but reactive nature of hydrazide when used as an enzyme substrate. Such substrates are targeted by disease activated protease because it contains a bond similar to the amide links of a peptide chain that are innately targeted by protease. Such cleavage action against the hydrazide substrate however releases the reactive hydrazine moiety of the substrate that in turn bonds to the protease enzyme causing its dysfunctional shutdown. This action stops incessant disease activity that holds cell maintenance systems at bay, and also halts the production of all peptides, viral coat protein, metastatic envelope protein, and such like that is necessary for disease activity and proliferation. The dysfunctional state of the protease system is also inductive of cell maintenance system action that the applicant suggests will scan nucleotide sequences to locate and remove damaged or disease altered DNA components, else the diseased cell is replaced or apoptoticly terminated. In either instance the disease is eradicated from the cell, or the diseased cell is eradicated from the system.

### BRIEF DESCRIPTION OF SEVERAL VIEW OF THE DRAWING

(Not applicable)

### DETAILED DESCRIPTION OF THE INVENTION

Distinguishing the Invention from Other Inventions:

The invention provides a safe and rapid way to shutdown disease activated protease in cells taken over by diseases as cancer, viral infections, and other disorders that are established in the genetic code of cells. Such diseases are believed to utilize similar metastatic mechanisms that are dependent on peptide controlled cell activities, and the production of protein coats or metastatic protein envelopes necessary for proliferation. It is believed that a disease activated protease enzyme is incessantly active and such activity doesn't pause at any time which would allow cell maintenance systems to scan disease altered nucleotide components needed to locate and eradicate such diseased altered DNA in the cell.

Such incessant activity that hides disease altered cells behind incessant activity is overcome by the inert hydrazide substrate mechanism. Such method shuts down cell activity which does allows cell maintenance systems to function to either locate and eradicate the disease code from the cell, or terminate the cell from the body. This compares to traditional HIV protease inhibitor drugs that can only inhibit in part such protein production by targeting disease specific receptors to interfere with the assembly of the peptide chains used by a disease active cell. However disease specific receptors exists in both disease infected cells and health cells. Because such choice of receptors are disease specific, a drug that targets HIV specific receptors would not have application for other type viral infections where the targeting of a different receptor site is needed. Also because the process affects diseased cells and non diseased cells alike, the metabolic interference of health cells presents a major problem. As such the protease inhibitor drug method is limited in scope to only one disease application and the receptor obstruction method causes serious metabolic problems and other untoward side effects for such method.

Such current protease inhibitor methodology is in stark contrast to the hydrazide substrate's benign method that provides a total shutdown of protein production provided when a single hydrazide molecule is targeted by protease cleavage. Instead of a method that targets receptors, the inert hydrazide substrate is targeted by disease activated protease. Such cleavage of the hydrazide substrate causes the protease to shutdown with complete cessation of all proteins produced by diseased cell activity. As such the inert hydrazide substrate is suitable to stop all disease action for all types of viral infections and other diseases that take control of the protease system. The hydrazide caused shutdown of protease directly halts all disease related protein produced by the diseased infected cell to provide the following:

[a] Such hydrazide shutdown stops all viral coat protein, and peptide signals that control cell functions for viral infections;

[b] Such hydrazide shutdown stops peptides responsible for cell division and growth to block reproduction and proliferation of all infectious microorganisms;

[c] Such hydrazide shutdown stops peptides responsible for cancerous cell division and peptide envelopes responsible for the metastasis of all types of cancer;

[d] and if for some unforeseen reason a cure for any diseases that produce toxic proteins should not be imminent, then the hydrazide shutdown of protease would quickly halt production of aberrant or excessive proteins characteristic of fatal diseases, especially as myeloma and Alzheimer's diseases provide examples.

The targeting of the hydrazide substrate is virtually a diseased cell mechanism. The continuous incessant production of protein by cells does not occur in cells of the healthy body. In a healthy cell proteins are only produced in response to systemic needs which are intermittent and occur only on an occasion of need. This difference explains how the hydrazide drug quickly becomes the target of incessant disease activated protease as cancer and viral diseases exemplify, that make continuous demand for protein products necessary for disease activity and proliferation. Such incessant disease activity largely depletes normal cell substrates used to build peptide chains needed by disease activity and as such the hydrazide substrate is essentially without competition pertaining to protease targeting of substrates when it is introduced. As such the hydrazide substrate is immediately targeted by cleavage action which requires only a single hydrazide molecule to be cleaved in order to causes the hydrazine moiety to attach to the enzyme which shuts down all protein production in a diseased cell. In a non-diseased cell the demand for continuous protein production does not exists and as such healthy cell shutdown by hydrazide cleavage is less likely to occur due to the accumulation of substrate resources in a cell that would compete with the hydrazide substrate.

Biological Mechanism of Action:

The inert hydrazide drug shuts down the protease enzyme as a result of the active hydrazine moiety that is set free by cleavage action which immediately attaches to the enzyme structure to provide the irreversible substrate action. The irreversible substrate design requires only that one hydrazide molecule be targeted by cleavage action to cause the complete shutdown of a viral replicating cell, or a dividing cancerous cell, or other disease system or condition that resides in the genetic code of cells.

The biological mechanism of the irreversible and lasting action of the inert hydrazide drug substrate involves the molecular attachment of the hydrazide drug's hydrazide moiety at a critical location on the protease enzyme's molecular structure. This can be explained as a result of the hydrazide's reactivity when set free by the protease enzyme's cleavage action during proteolytic cleavage of substrates. The hydrazide substrate is targeted because it has a “hydrazine-acid” bond in its molecule (R′NHNHCOR″) that appears as an “amino-acid” bond that comprises an amino acid link (R′NHCOR″) in a peptide chain or a substrate resource. When proteolysis action is attempted the cleavage of the hydrazide moiety immediately transfers the hydrazine bond to the protease enzyme. That attachment halts protease action as an obstruction to enzyme operation and also blocks further input of peptide substrates that supply the protein building process. As such the action serves to arrest all activity by the protease system and associated cell machinery linked to protease action whereby it remains inactive until cell maintenance processes discover the dysfunctional state and restores the cell with genetically correct or disease free components, or apoptotic termination of the cell occurs.

Process of Making and Using the Hydrazide Embodiment:

This invention is based on the active nature of hydrazine that irreversibly attaches to the protease enzyme to cause its shutdown. Such action is provided best by a hydrazide molecule where the reactive hydrazine component is supplied in an inert and unreactive molecule such that the exposed hydrazine terminal be rendered inert by addition of a substituent. Such is accomplished in an alkyl-hydrazide product, and in double acid hydrazide as maleic hydrazide represents. The process of making and using an inert hydrazide substrate drug is provided as follows in sufficient detail that a person skilled in the art can synthesize the same. In a formal sense, hydrazides are compounds of hydrazine and an organic acid in which the —OH of the acid is replaced by the hydrazino group (—NHNH2). The preferred embodiment proposed by this invention is provided by a alkyl-hydrazino group, or related substituents. Such carboxylic acid hydrazides are easily made simply by allowing the organic acid ester and the alkyl-hydrazine components to react. Iproniazid is an alkyl-hydrazide drug used by this applicant in this research because such drug has a known history of medical use as a MAOI antidepressant drug, and such drug or its analogs are easily made by several simple processes.

A hydrazide substrate drug of any chosen organic acid component and the alkyl hydrazide component are best prepared simply by allowing equal molar quantities of the acid ester and the alkyl-hydrazine compounds to react in a suitable solvent. Such alkyl hydrazides formed are weakly basic, essentially inert and unreactive in normal environments, and can be purified by crystallization using an appropriate mineral acid. The isopropylhydrazide component as needed to make iproniazid, or related alkyl hydrazides is best prepared from an alkyl bromide as isopropyl bromide exemplifies, and by using a 5-fold molar equivalent excess of aqueous hydrazine in an alkaline medium. The alkyl hydrazide derivative formed is easily salted out by addition of an excess of hydrochloric acid which causes a crystalline precipitate of the alkyl-hydrazine salt to settle.

Numerous variations of hydrazide preparations exist, and numerous types of hydrazides are possible that will provide a medically viable hydrazide substrate product. A novel example of this fact is illustrated by recent publications of a mysterious and unexplained cure for warts as provided simply by covering the wart with duct tape whereby the wart disappears within a few weeks or months. This can be explained as hydrazides and hydrazine as used in making polymers and adhesives as used to make duct tape. This applicant suggests that because warts are manifestations of viral infection of the skin, and viral infections are sensitive to hydrazides that the tape provides a topical application means for hydrazides. The hydrazides are emitted slowly by the tape sufficiently to eradicate the viral infection. The expected result of wart tissue no longer supported by a viral DNA program would be apoptotic termination of warts or cancerous growths which would disappear over time.

Best Modes for Using this Invention:

Any pharmacological preparation or means, including duct tape, that can introduce the hydrazide into a DNA altered or damaged cell is applicable. The nature of this discovery provides use by oral dosage forms with almost the same safety and ease of use as that provided by an over the counter remedial medication. It can be used for the most serious of diseases, to the most mild or insignificant of conditions. It is believed that diseases that can be effectively treated or cured by hydrazide therapy are diseases that share DNA with a host cell to produce a disease or a condition that is characterized by a specific protein, protein antigen, or other characteristic protein product not found in the healthy body. Such disease specific protein products are essentially indications that the genetic code has been altered by disease, or damaged by environment which has served to essentially alter or change the DNA programming. Viral altered genetic code and cancers are obvious examples, but the applicant believes many less infectious or malignant diseases, and conditions also result from genetic code degradation.

Many such conditions occur with age and are caused by free radical, radiation, or toxic substance type damage to nucleotide bodies that occur over time. If such damage occurs at a locations that serves as an encoded peptide sequence then the such related peptide template would reflect such damage by having an altered amino acid position at the corresponding location where such damage or alteration occurred. Such alterations would not stop, or affect protein production, and may not become apparent until similar damages accumulate in like cells to degrade biological regulation efficiency, or cause other diverse conditions. Such conditions could be as benign as “age spots” that are manifested on the skin, or as severe as schizophrenia, autoimmune disease, adult diabetes, or essentially any condition peculiar to that suffered more so by the elderly than by the young.

The inert hydrazide substrate shutdown of protein production has therapeutic benefits for such diseases and conditions that are synergistic at several levels. Such shutdown halts toxic protein production and systemic damages as exists with diseases as severe as myeloma and Alzheimer's disease. Secondly the cessation of protein results in a suspension of the proliferation, or metastatic transfer of such DNA encoded diseases or conditions to neighboring cells. Such metastatic transfer is the same as that illustrated by viral coat production and as cancer metastasis envelopes provide. And thirdly the dysfunctional inactive state produced by hydrazide shutdown allows cell maintenance action to intervene which scans and repairs altered or damaged genetic code found, else such cells are terminated by apoptotic processes. Essentially the method provides for the eradication of altered DNA programming whether it is provided by a serious disease or merely a condition attributed to environmental exposure caused by free radical, radiation, or toxic substance type damage.

Viral Infection and HIV/AIDS Therapy:

The Acquired Immune Deficiency Syndrome (AIDS) is caused by human T-lymphotropic retrovirus, also known as the human immunodeficiency virus, or HIV that principally infects the human T-4 lymphocyte cells. The retroviral genome is composed of RNA which is converted to DNA by reverse transcription where it takes control of the infected cell components to provide a replication process. Replication is dependent on the disease encoded protein and peptide products as the viral coat protein, peptides, maturation protein, various enzymes as RNA replicase subunit, and additional host cell protein products may illustrate.

Such disease dependency on protein makes viral disease vulnerable to protease system dysfunction and as such the protease system becomes a viable target for inhibiting such disease action and replication. It is because protein production is vital to viral disease that much attention has focused on the development of HIV protease inhibitor drugs as a means to slow such protein and peptide products as a means to slow disease progression. Indeed all viral infections are dependent on disease activated protease systems and as such the protease system represents a viable target that can be used to control such diseases as current protease inhibitor drugs illustrate. The hydrazide substrate method however shuts down all protein products produced by an HIV infected cell whereas protease inhibitor drugs slow the production of such proteins but cannot shut it off as the hydrazide substrate method accomplishes.

The inert hydrazide substrate method provides a reaction to a viral activated protease system that provides a dysfunctional shutdown of the protease system controlled by viral infection. In a private study iproniazid was used to eradicate an influenza infection essentially overnight and an advanced HIV/AIDS infection within a few days time. The therapeutic method was essentially the same as that for using iproniazid as an antidepressant MAOI type drug. There was a concern that due to HIV damage to the immune system that the immune system may require time to recover beyond what may appear to have been effective to eradicate the disease. As such it would appear that several weeks of continued therapy would be sufficient to satisfy the need required to stop any infection from reemerging. Such antiviral action can be evidenced to exist against any and all viral diseases by viral screening using the inert hydrazide substrate provided by iproniazid against a representative sampling of different viruses. This Iproniazid phosphate also exists in some laboratories as used to preserve tissue homogenates against oxidase degradation which in effect also prevents protease enzyme degradation of the medium also.

Prion Infection Therapy:

Prion infections are similar to viral infections in many respects but are lacking a nucleic acid component outside the cell. The prion protein structure nevertheless appears to be able to use existing nucleic acid components to encode its protein structure in cell DNA such that the cell can replicates prion protein particles afterwards. Because the inert hydrazide substrate shuts down any active protease production including that which replicates prion particles, such provides a method to suppress prion type infections at least to a limited extent, to reduce the amount of prion protein being replicated by such infected cell.

Hydrazide substrate therapy may not provide a method capable of eradicating the disease since any shutdown and subsequent cell repair process would likely not recognize or find any abnormalities in the genetic code to eradicate. This is because the prion mechanism appears to be related to a mechanism of protein as it pertains to memory, where memory is encoded in protein as a normal CNS cell process. Such memory mechanism would require that such protein have a means existing in the genetic code, to be encoded in nucleic acid serving as a replicating means to share such protein with other cells. As such it is believed to be unlikely that such process connected to innate memory processes as may be initiated by a prion particle would manifest any abnormal appearance in cell DNA that could be detected by cell maintenance systems that could repair such problem or cause cell termination to occur.

However, whether cell maintenance systems can eradicate nucleic acid components that encode the prion germ, or can terminate the cell itself following hydrazide therapy cannot be known with certainty until tested on animal models. As such if the cell is terminated by the hydrazide substrate then such provides a curative process, and if the cell is not terminated it nevertheless provides a treatment process to slow the disease progression. In a treatment process the continual consistent use of hydrazide therapy would serve to shutdown protease action in much the same fashion that MAOI use slows down oxidase enzymes destruction. As such the hydrazide substrate action would slow progression of the prion infection if such use does not eradicate the disease.

Cancer and Related Disease Therapy:

Cancerous growth, and metastasis is blocked by action of the hydrazide substrate as such shuts down the protease enzyme responsible for producing essential protein products required by the disease. The protease system produces peptide products that control and initiate cancerous cell division and the enzyme also provides metastatic protein envelopes necessary to transmit the cancer germ encoded in nucleic acids to uninfected cells. Cancer is similar to viral infections in respect that its mechanism is encoded into nucleic acid that redirects cell action to provide cancerous cell division that spreads to cells through a metastatic mechanism believed to be similar to that of an RNA virus.

As such cancers cell division and metastatic transfer is dependent on protease action and is mediated directly or indirectly through the release of active peptide signals, and as such it is dependent on a number of protein or peptides needed to regulate biological conditions and cell activities before cell division or metastasis can occur. Hydrazide substrate treatment shuts down all such protein and peptide signals and hence stops cancerous cell division and metastasis. It is believed that a lasting cure would result from cell maintenance activity that would eradicate the cancer germ existing in the genetic code, and that the sterilized diseased cell itself would eventually be terminated by apoptotic process. In other words if a cancerous growth exists when hydrazide therapy begins that it will also exist for a period of time after the cancer related genetic code is eradicated by cell maintenance system action. It is probable that apoptotic breakdown of cancer cell will occur in a timely fashion to remove all traces of such disease within months which could easily be determined using iproniazid phosphate testing on animal models. Iproniazid exists in some laboratories as uses to preserve tissue homogenates from oxidase degradation.

Microorganism Infection Uses:

The hydrazide drug prevents peptide productions that controls cell division, growth, reproduction or proliferation of infectious organisms as bacterial, fungal, protozoal, metazoa, and other microorganism types. The effects of the hydrazide substrate on microorganisms as a means to stop proliferation is easily demonstrated using basic microbiology techniques. The difference being that hydrazides do not injure or kill microorganisms, it only prevents cell division, growth, and proliferation of the organisms. As such it provides a means to stop an infection or its proliferation, but does not have any means to kill such organism.

As such, and from a therapeutic point of view, hydrazides may not be impressive to terminate an infection caused by some microorganisms, but it has an unrecognized benefit to hold reproduction and proliferation in check while trying antibiotics with questionable efficacy. A second important use would be to insure that the microorganism has no progeny to pass on antibiotic resistance traits to successive generations. Antibiotic resistant organisms have become a major problem due to world population levels that are increasingly dependent on a limited number of antibiotic drugs. Such problem is exacerbated by the unnecessary and frivolous use of antibiotics in the livestock industry. Such problems and abuses would be negated if a hydrazide drug were prescribed concurrent with antibiotic use, or added to animal antibiotic preparations, or simply added in minimal amounts to animal feed. Such would prevent livestock losses due to disease and also provide prophylaxis protection for livestock.

Biological Regulation Disorders:

The hydrazides substrate mechanism provides use to treat biological control disorders that result from minor changes, or minor alterations of genetic code that can impact a given amino acid position of the peptide template which negates the action of bioactive peptides, hormones, and prostaglandin products derived from such corrupted template. Such peptide products control biological processes that depend on a precise peptide coding sequence needed to address or link such action to a peculiar receptor or effector cell that in turn supplies a systemic need, or biological regulation process. Treatment and a cure is possible due to the hydrazide drug mechanism able to induce cell maintenance scans of nucleic acid components where aberrant protein products are cause by template receptor damages.

Such template damages accumulate with age and is believed to be the cause of many age related maladies. Adult onset diabetes provides an example easily understood where a general diminishing of systemic control capability is seen with age that may illustrate a condition where more and more cells that produce the insulin hormone becomes damaged such that an error is encoded into the spelling sequence of such peptide template. Such peptide template damage that causes such diminishing effectiveness of biological processes with age is believed to be due to free radical, radiation, or toxic substance type damages of the nucleotide which accumulates with age. If such nucleotide occurs within a template coded sequence, such template alterations results in the production of peptide product having an altered peptide sequence, or a broken peptide chains, resulting in an a product having little or no biological activity as a bioactive peptide, or hormones, or a prostaglandin product.

The effects of altered bioactive peptides, hormones, and prostaglandin products could be manifested as conditions of endocrine obesity, schizophrenia, autoimmune disease, anorexia, collagenous disease, colitis, severe neurotic pain or increased sensitivity to pain, angina pectoris, hypertension, psoriasis, immune dysfunction, high cholesterol, glaucoma, some cravings and addictions, and countless additional possibilities that are subject to benefits of hydrazide substrate therapy. Since such biological regulation disorders can also have disease origins the diagnosis is further complicated. The therapeutic benefits of inert hydrazide substrate therapy however is effective irregardless of cause as it shuts down such aberrant peptide productions and once the cell is shutdown there exists a probability that cell maintenance scans of the nucleic acid sequence will locate damaged components and replace such with damage free cell replacements. Alternatively it is believed such cells that cannot be repaired are terminated or replaced through cell division.

Alzheimer's Disease Therapy:

There exists a number of conditions and diseases that produce aberrant and mutant protein products as Alzheimer's disease, multiple myeloma, multiple sclerosis, Parkinson's disease, which appear as a cluster of protein or plaque deposits. The genesis of such diseases likely resemble the damaged template model described that appear with age having characteristics of an RNA virus or a cancer. The exact mechanism at fault to cause Alzheimer's disease is not known but if such disease has not been caused by corruption to a peptide alignment template then damage to biological controls that induce protease system activity could explain a runaway action where a natural protein is replicated in excess.

Irregardless of cause excesses of a protein product that cannot be used must accumulate around the cell, or cells where it is generated. Such protein excess would present a disposal problem which would appear as an abundance of protein plaque with tangled peptide strands or lumps that largely deposit around the locations where it is generated in the nervous system. The interference and crowding of neurons by such plaque would eventually lead to atrophy of surrounding tissue causing dementia as is characteristic of Alzheimer's disease. However it is believed that the runaway protease system action causing the problem could easily be arrested by the hydrazide drug mechanism thus halting disease action and allowing cell maintenance systems to recognize the dysfunctional state, locate the damage, and replace faulty components. In the alternative it is believed that such cells would be terminated.

However the victim of Alzheimer's disease would hardly notice any change if a cure is effected as the atrophy and impending damage to additional neuron growth would remain and would need to be removed to avoid further damage and to provide space for new neuron. and dendrite growth to exist. As such the effects of the disease would remain somewhat even though the cause of the disease is eradicated. Full recover would likely require additional novel means to assist in plaque removal as using digestive enzymes and immune system adjuvants. Additionally nerve growth stimulants should stimulate new nerve growth and dendrite development that would hasten recovery of lost mental functions suffered by such victims.

Multiple Myeloma Therapy:

Multiple Myeloma provides a second possible scenario to explain a false active protease production scenario. This disease can be explained where the nature of the damage occurs in a way to alter or obstruct a position of the encoded peptide alignment template sequence as it exists in genetic code. The resulting template would in turn allow a protein chain to be assembled up to a position where the encoded template was altered or obstructed and as such the RNA product could be a peptide chain shorter, or altered at the affected position, or possibly simply appear as a broken chain. The specific protein need as existed before disease infected the cell would continue to exist at the systemic level, and as such systemic signals that activate protease production would occur perpetually to request the needed protein keeping such process active. The proteins produced are apparently able to serve as a protective metastatic envelope sufficiently to transfer the altered genetic material to infect nearby cells. As such the disease progresses and the need for systemic correct protein increases due to the increasingly smaller number of cells existing that can provide such unaltered protein product.

Based on such type of scenario the consequence of a relentless disease action that produces an aberrant or broken peptide chain could be undermining of organs or structures that normally uses such product. Such excess production of shorter defective protein being produced would cause damage and present disposal problems for the kidney as may be in character for multiple myeloma. The incessant disease activated protease system producing such aberrant protein products would easily be shutdown by the hydrazide substrate which could be a life saving action since it stops the toxic protein production immediately without untoward effects. It is believed that the disease would be placed in full remission due to hydrazide substrate shutdown that is inductive of cell maintenance action. Otherwise the static sell condition is believed to trigger apoptotic programs existing in cell chromosomes that terminate such cells. The hydrazide substrate efficacy as an aberrant protein disease treatment or cure, such as myeloma exemplifies, could easily be determined using animal models based on iproniazid or other impromptu synthesis of any similar alkyl hydrazide molecule.

Hydrazide to Treat Obesity:

Many additional applications are possible for hydrazide drug therapy that becomes apparent by screening medical texts for diseases or health problems associated with protein, or otherwise characterized by emission of a disease specific protein or antigen. Probably all infectious diseases and cancers are subject to hydrazide therapy and many novel uses outside these areas likewise exist. For example there exist endocrine obesity causes and mental conditions that affect eating behavior due to peptide encoded errors that could account for absence of biological signals that communicate appetite or cravings, or the lack thereof. Such peptide sequence errors were discussed under the biological regulation disorders topic which is applicable here to explain over indulgence and addictive behavior due to an absence of correctly addressed peptides needed to communicate satisfaction or related biological messages.

Additionally a treatment from a different approach as it obtains to obesity is possible based on the fact that fat cell increases could be halted somewhat by the hydrazide drug mechanism as protein and peptide signals are also necessary to trigger or induce fat cell division necessary to generate extra fat storage space. Without increased numbers of fat storage cells such fat must be used or discharged as waste.

Hydrazide to Prevent Age Effects and Cell Death:

The length or duration of life varies from creature to creature and has been shown to be a result of a genetically programmed process such that a creature will eventually die of old age if an untimely demise does not otherwise intervene. The process is evidenced to exist at the cellular level as genetic coded programs passed on to progeny as program codes existing in the chromosomes. Such cell death programs provide for cell apoptosis to be triggered or induced by a summation of events. This cell death program is believed to be initiated by age and other events that toll stress, disease, injury, and essentially any deprivation of life sustaining substance at levels less than what would be required to otherwise be a cause of cell death.

In a similar vein the breakdown of body proteins and muscle tissue also occurs with age as a systemic process connected with such programs that are induced or transmitted by peptide or polypeptide signals and enzyme processes. Because the hydrazide substrate halts protease and the production of peptide signals as well as providing a direct action to shutdown proteolysis of protein in muscle, the hydrazide shutdown of active protease provides a means to halt, or hold back such processes from occurring. As such hydrazide therapy provides the means to withhold biological changes during times of stress and to hold off aging effects and even cell death during times of crisis, stress, or injury. This hydrazide substrate method would also improve chances for recovery from disease or injury by rescuing and preventing apoptotic termination of cells brought on by chromosomal programs that would otherwise be survivable. Such protection for cells in jeopardy would thereby provide time for a recovery process, or for medical intervention needed to resuscitate such cells that otherwise would be terminated when asphyxia is threatened as near drowning, heart attacks, stroke, and when other traumas places cells in jeopardy of apoptotic termination.

Hydrazide as Prophylactic Against Disease:

Prophylactic protection against disease is a need that exists concurrent with and following some drugs as corticosteroid and immunodepressant drug therapy, and can be satisfied by using the inert hydrazide substrate method as a prophylaxes drug. Such need also exists following exposure to an infectious disease, or merely as a safeguard when infectious disease exposure threatens. The principal hydrazide substrate drug used in this research was iproniazid which has been used as an antidepressant drug for over 50 years in some countries abroad. And because the hydrazide type MAOI drug is essentially side effect free following long periods of use as an antidepressant, and because there exists no tolerance mechanism to preclude such use as would apply to continuous antibiotic use, hydrazide drug use to provide prophylactic protection against diseases provides one more powerful application for hydrazide drug use. Such prophylactic use could reasonably continue through life to provide a better quality of longer disease free life in addition to benefits that would prevent aging or apoptosis termination of cells as occurs with age or trauma.

Alternative Embodiments and Precursors Thereof:

The distilled essence of this invention can be summarized as a method using hydrazine to shutdown protease enzymes as results from protease cleavage action. Its value is due to the fact that it provides the means to shutdown disease activated protease to halt disease action for which there exists no satisfactory treatments. The embodiment preferred by this invention is to package such hydrazine inside a hydrazide molecule to provide maximum efficacy while eliminating the toxic effects of an exposed hydrazine terminal. The simple functional group based on a acid and hydrazine bond on which the discovery is based can exist in many forms. Hydrazine products alone provide the effects of a hydrazide as they are altered in vivo by acetylation thus converting them into the hydrazide embodiment, but such route is very toxic and damaging to the system.

Additionally viable embodiments of this invention can be provided using an unlimited variety of organic acid products including cyclic, dicarboxylic, and other forms. Such molecules can also be provided in modified forms as sulfonyl hydrazides, thiohydrazide analogs and such like that provide a viable “acid-hydrazine” bond capable of providing protease cleavage targeting, and hence will provide the hydrazide substrate mechanisms described in this disclosure. The reaction between aldehydes with hydrazine providing hydrazones also provides a viable protease inhibitor product due to oxidase enzyme transformation of aldehydes into organic acids. Like products provided by ketones and hydrazine are also subject to in vivo acetylation to again provide a hydrazide compound which encompasses the spirit of this invention. As such the present invention should not to be limited by the specific embodiment described as many changes, modifications, and substitutions in addition to the precursor chemical forms can provide viable protease inhibitor products without departing from the spirit of the present invention.

Methods to treat disease using protease inhibitor drugs were non existent until HIV/AIDS appeared. The search for a method to slow or stop protein used by the virus stimulated abundant research to find a means to halt or suppress protein production. The only means discovered capable of providing a useful protease inhibitor product is the common protease inhibitor drugs which at its very best has very limited efficacy and untoward side effects. However such method is nevertheless better than nothing as such drugs have extended the lives of many HIV/AIDS victims. The technology has since improved and it is recognized that many diseases exist that can be treated by such conventional protease inhibitor methods, and the most recent example known to the applicant is the protease inhibitor methods being developed for multiple myeloma.

However the present invention provides a superior method where one molecular design is effective for all diseases subject to the protease shutdown method. Such method halts disease activated protease to stop all protein, rapidly, without untoward effects but with additional benefits unexpected for such discovery. This invention also provides new methodology to essentially stop only disease activated protease, and such method has application against a wide range of diseases and medical conditions too numerous to enumerate. Secondly the method halts aberrant and toxic proteins as is characteristic of many diseases and conditions which further increase the scope of novel uses for this discovery. Thirdly, the method is inductive of cell maintenance removal or eradication of disease altered and damaged genetic code which use eradicates the cause of many diseases and conditions not considered as diseases. As such the compounded scope of medical uses would require a thick book to address in exhaustive fashion. As such the present invention is not intended to be limited by the examples given as many additional uses will become apparent to those skilled in the art without departing from the spirit and scope of the invention.

Information provided herein is believed to be correct even though some aspects of this disclosure provides workable theory to explain how and why this invention does what it does. As such the applicant does not wish to be bound by theory as he believes the information as presented will better convey an understanding of theory, operation, and uses to persons skilled in the art.

Definitions:

The term “protease” and “proteasome” as used herein refers to the protease system comprising enzymes and RNA components necessary to assemble proteins, peptides, hormones, and prostaglandin products. The term “hydrazide substrate” unless otherwise stated refers to the inert hydrazide substrate embodiment of this invention which requires only a hydrazide molecule with a substituent attached to the exposed hydrazine terminal of the hydrazide molecule necessary to negate the reactive and toxic effects of hydrazine. The term “hydrazide substrate drug” as used herein refers to any alkyl-hydrazide drug, or other hydrazide forms with a substituent that is a necessary addition to the hydrazine terminal to negate toxic effects produced by hydrazine. Existing hydrazide substrate drugs that qualify under this definition are iproniazid (Marsilid), isocarboxazid (Marplan), and nialimide (Niamid).

The applicant claims the following:

1. A compound consisting of a hydrazide molecule as a part of its construction, where the carboxylic acid moiety of said hydrazide molecule is provided by any organic acid, and where the hydrazine moiety of said hydrazide molecule is provided by hydrazine.

2. The compound according to claim 1, where said hydrazine moiety has a substituent other than hydrogen.

3. The compound according to claim 1, where said carboxylic acid moiety is provided by nicotinic acid and where said hydrazine moiety is provided by isopropyl hydrazine.

4. The compound according to claim 1, to provide pharmaceutical use as a protease inhibitor drug that shuts down disease activated protease enzymes.

5. A method of treatment for a condition, disorder, or disease by administering to a patient in need thereof an effective amount of a pharmaceutical compound according to claim 1.

6. The method according to claim 5 where said disease is a viral infection which includes but not limited to a member belonging to arenaviridae, bunyaviridae, caliciviridae, coronaviridae, flaviviridae, hepadnaviridae, herpesviridae, orthomyxoviridae, papovaviridae, paramyxoviridae, parvoviridae, picornaviridae, poxviridae, reoviridae, retroviridae, rhabdoviridae, and togaviridae families that includes members as adenovirus, hepatitis type A, hepatitis C, hepatitis B, herpes simplex type I, herpes simplex type II, human T-lymphotropic retrovirus III, human immunodeficiency virus, influenza, influenza, measles virus, mumps virus, papilloma virus, papova virus, rhinovirus, rubella virus, SARS crown virus and varicella whereby said dysfunctional shutdown of protease provides a cessation of peptides and viral coat protein needed for replication and is inductive of cell maintenance disease eradication processes.

7. The method according to claim 5 where said disease is a prion infection which includes but not limited to Creutzfeldt-Jakob Disease, Gerstmann-Straussler-Scheinker Disease, kuru, and bovine spongiform encephalopathy whereby said dysfunctional shutdown of protease provides a cessation of prion protein production and is inductive of cell maintenance disease eradication processes.

8. The method according to claim 5 where said disease is cancer which includes but not limited to acoustic neuroma, acute granulocytic leukemia, adenocarcinoma, adrenal cortex carcinoma, angiosarcoma, astrocytoma, basal cell carcinoma, bile duct cancer, bladder carcinoma, breast cancer, bronchogenic carcinoma, cervical endometrial carcinoma, cervical carcinoma, cervical hyperplasia, chordoma, choriocarcinoma, chronic sarcoma, colon carcinoma, craniopharyngioma, cystadenocarcinoma, endotheliosarcoma, ependymoma, epithelial carcinoma, esophageal carcinoma, thrombocytosis, fibrosarcoma, glioma, hairy cell pancreatic cancer, neck carcinoma, hemangioblastoma, hepatoma, Kaposi's sarcoma, leiomyosarcoma, leukemia, liposarcoma, lung carcinoma, Hodgkin's disease, macroglobulinemia, malignant hypercalcemia, carcinoid carcinoma, pancreatic insulinoma, malignant melanoma, medullary carcinoma, medulloblastoma, melanoma, meningioma, multiple myeloma, mycosis fungoides, myxosarcoma, neuroblastoma, oligodendroglioma, genitourinary carcinoma, osteogenic sarcoma, ovarian carcinoma, pancreatic carcinoma, papillary adenocarcinomas, pinealoma, polycythemia mesothelioma, primary macroglobulinemia, primary brain carcinoma, prostatic carcinoma, renal cell carcinoma, retinoblastoma, leukemias, rhabdomyosarcoma, sarcoma, sebaceous gland carcinoma, seminoma, skin cancer, small cell lung carcinoma, soft-tissue lymphangiosarcoma, squamous cell carcinoma, stomach granulocytic leukemia, sweat vera, synovioma, testicular tumor, testicular carcinoma, and thyroid carcinoma whereby said dysfunctional shutdown of protease halts protein and peptide production necessary for cancerous cell division, growth, and metastasis, and is inductive of cell maintenance disease eradication processes.

9. The method according to claim 5 where said condition is caused by protease production of toxic or aberrant proteins that includes but not limited to multiple myeloma, Alzheimer's disease, Parkinson's disease, and other disorders whereby said dysfunctional shutdown of protease halts toxic protein production and is inductive of cell maintenance action to restore the peptide genetic encoding sequence or to terminate such cell.

10. The method according to claim 5 where said disorder is a loss of systemic control or biological regulation processes caused by slight alterations in proteins, peptides, hormones, or prostaglandin products as used to communicate or control biological regulation processes manifested as conditions that includes but not limited to acne vulgaris, adenomatous intestinal polyposis, adenosine deaminase deficiency, alcoholism and other addictions, anemia, angina pectoris, anorexia, arteriosclerosis, carbonic anhydrase deficiency syndrome, classical phenylketonuria, colitis, collagenous diseases, Crohn's disease, cystic fibrosis, diabetes mellitus, Gaucher's disease, glaucoma, glucose-6-phosphste dehydrogenase deficiency syndrome, high cholesterol, hypercholesterolemia, hypersarkosinemia, hypertension, spontaneous tumors, malignant melanoma, multiple sclerosis, muscular dystrophy, obesity, osteopetrosis, psoriasis, schizophrenia, severe or neurotic pain, spondylitis, t-cell immunodeficiency, triosephosphate isomerase deficiency syndrome, and ulcerative colitis whereby said dysfunctional shutdown of protease is inductive of cell maintenance action to restore peptide sequence integrity responsible for such systemic communications breakdown.

11. The method according to claim 5 wherein said condition is a microorganism infection which includes infections caused by organisms belonging to absidia, acanthamoeba, acanthocephala, aspergillus, balantidium, basidiomycota, blastomyces, candida, cestoda, cestodes, chaetognatha, cladosporium, coccidioides, comycota, cryptococcus, cycliophora, deutoeromycota, entamoeba, gastrotricha, geotrichum, histoplasma, leishmania, leptomyxida, mucor, naegleria, nematoda, paracoccidioides, phialophora, plasmodium, platyhelminthes, pneumocystis, pseudoallescheria, rhizopus, rhodotorula, rotifera, sporothrix, torulopsis, toxoplasma, trematoda; aschelminthes, trematodes, trypanosoma, xylohypha, and zygomycota whereby said dysfunctional shutdown of protease halts peptide production necessary for cell division, reproduction, and proliferation of said organisms.

12. The method according to claim 5 where said disease is a bacterial infection caused by any member of the species related to actinomycosis, anthrax, bartonellosis, borreliosis, brill-zinsser, brucellosis, campylobacter, chlamyldial, cholera, clostridial bacteroides, ehrlichiosis, enterobacteriaceae, erysipelothricosis, gonorrhea, hemophilus, legionella, leptospirosis, listerosis, melioidosis, mycoplasma, neisseria, nisseria, nocardiosis, noncholera vibrio, plague, pneumoccal, pseudomonas, rickettsioses, salmonella, shigellosis, spirochetes, staphyloccal, steptoccal, streptbacillus, syphilis, treponematoses, and tulamremia whereby said dysfunctional shutdown of protease halts peptide production necessary for cell division, reproduction, and proliferation of any member of said species.

13. The method according to claim 5 wherein said condition is a need to prevent antibiotic resistant strains of organisms from evolving as a result of antibiotic use whereby said dysfunctional shutdown of protease that is innate to said organisms halts peptide production necessary for cell division, reproduction, and proliferation of progeny that could pass on antibiotic or drug resistant traits to successive generations.

14. The method according to claim 5 wherein said condition is a need to prevent and infection from worsening while testing antibiotic of unknown efficacy whereby said dysfunctional shutdown of protease that is innate to said organisms halts peptide production necessary for cell division, reproduction, and proliferation.

15. The method according to claim 5 to prevent disease or illness from occurring by providing prophylaxis treatment whereby such use serves to halt disease action before it can establish sufficiently to cause systemic affects or damage.

16. The method according to claim 5 where said condition is systemic induced cell death or apoptosis, caused by triggering of innate genetic programs existing in the chromosomes of cells caused by injury, age, or stresses, and as can result from hypoxia or ischemia, whereby said dysfunctional shutdown of protease provides a cessation of peptide signals necessary to induce apoptotic destruction of endangered cells.

17. The method according to claim 5 where said disorder is obesity or weight control whereby said dysfunctional shutdown of protease provides a cessation of peptides that induce fat cell division thereby causing the excretion of lipids that cannot be stored in the existing population of fat cells.

18. The method according to claim 5 where said disorder is an inability for systemic biological regulation processes to communicate feelings of satisfaction or hunger based on biological needs such that abusive behavior as over eating, over indulgence, or addictive behavior results, whereby said dysfunctional shutdown of protease is inductive of cell maintenance action to restore peptide sequence integrity responsible for such systemic communications breakdowns.

19. A process discovered as provided by a protease enzyme reaction to a hydrazide substrate
consistent with hydrazide substrate targeting by protease enzyme cleavage action,
where said cleavage action releases an active hydrazine radical,
where said hydrazine radical attaches irreversibly to the protease enzyme,
where said protease enzyme is rendered dysfunctional and inactive,
where said protease dysfunction halts the production of disease associated proteins,
where said dysfunctional state induces cell maintenance action,
where said cell maintenance action restores healthy cell operation,
where a disease free condition is thereby provided.

20. A method for providing a hydrazide substrate, protease enzyme inhibitor type therapeutic action by using any one of the existing hydrazide substrate, oxidase enzyme inhibitor type drugs which have identical therapeutic action exemplified by iproniazid, isocarboxazid, and nialimide which have a 50 year history of safe medical use.
